(19) United States
(12) Patent Application Publication
(10) Pub. No.: US 2008/0009782 A1
(43) Pub. Date: Jan. 10, 2008

(54) Methods and Devices for Transdermal Electrotransport Delivery of Lofentanil and Carfentanil

(75) Inventors: Robert M. Gale, Los Altos, CA (US); Rama Padmanabhan, Los Altos, CA (US); Joseph B. Phipps, Sunnyvale, CA (US)

Correspondence Address: DIEHL SERVILLA LLC, 77 BRANT AVE, SUITE 210, CLARK, NJ, 07066, US

(73) Assignee: ALZA Corporation, Mountain View, CA (US)

(21) Appl. No.: 11/768,569

(22) Filed: Jun. 26, 2007

Related U.S. Application Data
(60) Provisional application No. 60/806,048, filed on Jun. 28, 2006.

(51) Int. Cl. A61N 1/30

(52) U.S. Cl. 604/20

(57) ABSTRACT

Electrotransport drug delivery devices, systems and methods for delivery of lofentanil or carfentanil are disclosed. The lofentanil or carfentanil may be provided as a water soluble salt (e.g., lofentanil or carfentanil hydrochloride), such as in a hydrogel formulation. A transdermal, electrotransport delivered dose of lofentanil or carfentanil is provided which is sufficient to induce analgesia in (e.g., adult) human patients suffering from chronic, acute and/or breakthrough pain.

### CROSS-REFERENCE TO RELATED APPLICATIONS

This application claims the benefit of priority under 35 U.S.C. § 119(e) to U.S. Provisional Application Ser. No. 60/806,048, filed Jun. 28, 2006, which is incorporated herein by reference.

### FIELD OF THE INVENTION

The invention relates generally to electrotransport drug delivery. Specifically, the invention relates to devices, systems and methods for electrotransport delivery of lofentanil and carfentanil.

### BACKGROUND OF THE INVENTION

The transdermal delivery of drugs, by diffusion through the epidermis, offers improvements over more traditional delivery methods, such as subcutaneous injections and oral delivery. Transdermal drug delivery avoids the hepatic first pass effect encountered with oral drug delivery. Transdermal drug delivery also eliminates patient discomfort associated with subcutaneous injections. In addition, transdermal delivery can provide more uniform concentrations of drugs in the bloodstream of the patient over time due to the extended controlled delivery profiles of certain types of transdermal delivery devices. The term “transdermal” delivery broadly encompasses the delivery of an agent through a body surface, such as the skin, mucosa, or nails of an animal.

The skin functions as the primary barrier to the transdermal penetration of materials into the body and represents the body's major resistance to the transdermal delivery of therapeutic agents such as drugs. To date, efforts have been focused on reducing the physical resistance or enhancing the permeability of the skin for the delivery of drugs by passive diffusion. Various methods for increasing the rate of transdermal drug flux have been attempted, most notably using chemical flux enhancers.

Other approaches to increase the rates of transdermal drug delivery include the use of alternative energy sources such as electrical energy and ultrasonic energy. Electrically assisted transdermal delivery is also referred to as electrotransport. The term “electrotransport” as used herein refers generally to the delivery of an agent (e.g., a drug) through a patient's membrane, such as skin, mucous membrane, or nails. The delivery is induced or aided by application of an electrical potential. For example, a beneficial therapeutic agent may be introduced into the systemic circulation of a human body by electrotransport delivery through the skin. A widely used electrotransport process, electromigration (also called iontophoresis), involves the electrically induced transport of charged ions. Another type of electrotransport, electroosmosis, involves the flow of a liquid, which liquid contains the agent to be delivered, under the influence of an electric field. Still another type of electrotransport process, electroporation, involves the formation of transiently-existing pores in a biological membrane by the application of an electric field. An agent can be delivered through the pores either passively (i.e., without electrical assistance) or actively (i.e., under the influence of an electric potential). However, in any given electrotransport process, more than one of these processes, including at least some “passive” diffusion, may be occurring simultaneously to a certain extent. Accordingly, the term “electrotransport”, as used herein, should be given its broadest possible interpretation so that it includes the electrically induced or enhanced transport of at least one agent, which may be charged, uncharged, or a mixture thereof, whatever the specific mechanism or mechanisms by which the agent actually is transported.

Electrotransport devices use at least two electrodes that are in electrical contact with some portion of the skin, nails, mucous membrane, or other surface of the body. One electrode, commonly called the “donor” electrode, is the electrode from which the agent is delivered into the body. The other electrode, typically termed the “counter” electrode, serves to close the electrical circuit through the body. For example, if the agent to be delivered is positively charged, i.e., a cation, then the anode is the donor electrode, while the cathode is the counter electrode which serves to complete the circuit. Alternatively, if an agent is negatively charged, i.e., an anion, the cathode is the donor electrode and the anode is the counter electrode. Additionally, both the anode and cathode may be considered donor electrodes if both anionic and cationic agent ions, or if uncharged dissolved agents, are to be delivered.

Furthermore, electrotransport delivery systems generally require at least one reservoir or source of the agent to be delivered to the body. Examples of such donor reservoirs include a pouch or cavity, a porous sponge or pad, and a hydrophilic polymer or a gel matrix. Such donor reservoirs are electrically connected to, and positioned between, the anode or cathode and the body surface, to provide a fixed or renewable source of one or more agents or drugs. Electrotransport devices also have an electrical power source such as one or more batteries. Typically, at any one time, one pole of the power source is electrically connected to the donor electrode, while the opposite pole is electrically connected to the counter electrode. Since it has been shown that the rate of electrotransport drug delivery is essentially proportional to the electric current applied by the device, many electrotransport devices typically have an electrical controller that controls the voltage and/or current applied through the electrodes, thereby regulating the rate of drug delivery. These control circuits use a variety of electrical components to control the amplitude, polarity, timing, waveform shape, etc. of the electric current and/or voltage supplied by the power source. See, for example, McNichols et al., U.S. Pat. No. 5,047,007.

To date, commercial transdermal electrotransport drug delivery devices (e.g., the Phoresor, sold by lomed, Inc. of Salt Lake City, Utah; the Dupel lontophoresis System sold by Empi, Inc. of St. Paul, Minn.; the Webster Sweat Inducer, model 3600, sold by Wescor, Inc. of Logan, Utah) have generally utilized a desk-top electrical power supply unit and a pair of skin contacting electrodes. The donor electrode contains a drug solution while the counter electrode contains a solution of a biocompatible electrolyte salt. The power supply unit has electrical controls for adjusting the amount of electrical current applied through the electrodes. The “satellite” electrodes are connected to the electrical power supply unit by long (e.g., 1-2 meters) electrically conductive wires or cables. The wire connections are subject to disconnection and limit the patient's movement and mobility. Wires between electrodes and controls may also be annoying or uncomfortable to the patient. Other examples of desk-top electrical power supply units which use “satellite” electrode assemblies are disclosed in Jacobsen et al., U.S. Pat. No. 4,141,359 (see FIGS. 3 and 4); LaPrade, U.S. Pat. No. 5,006,108 (see FIG. 9); and Maurer et al., U.S. Pat. No. 5,254,081.

More recently, small self-contained electrotransport delivery devices have been proposed to be applied to the skin, sometimes unobtrusively under clothing, for extended periods of time. Such small self-contained electrotransport delivery devices are disclosed for example in Tapper, U.S. Pat. No. 5,224,927; Sibalis et al., U.S. Pat. No. 5,224,928; and Haynes et al., U.S. Pat. No. 5,246,418.

There have recently been suggestions to utilize electrotransport devices having a reusable controller which is adapted for use with multiple drug-containing units. The drug-containing units are simply disconnected from the controller when the drug becomes depleted and a fresh drug-containing unit is thereafter connected to the controller. In this way, the relatively more expensive hardware components of the device (e.g., batteries, LED's, circuit hardware, etc.) can be contained within the reusable controller, and the relatively less expensive donor reservoir and counter reservoir matrices can be contained in the single use/disposable drug-containing unit, thereby reducing the overall cost of electrotransport drug delivery. Examples of electrotransport devices comprised of a reusable controller, removably connected to a drug-containing unit are disclosed in Sage, Jr. et al., U.S. Pat. No. 5,320,597; Sibalis, U.S. Pat. No. 5,358,483; Sibalis et al., U.S. Pat. No. 5,135,479 (FIG. 12); and Devane et al., UK Patent Application 2 239 803.

In further development of electrotransport devices, hydrogels have become particularly favored for use as the drug and electrolyte reservoir matrices, in part, due to the fact that water is the preferred liquid solvent for use in electrotransport drug delivery due to its excellent biocompatiblity compared with other liquid solvents such as alcohols and glycols. Hydrogels have a high equilibrium water content and can quickly absorb water. In addition, hydrogels tend to have good biocompatibility with the skin and with mucosal membranes.

Of particular interest in transdermal delivery is the delivery of analgesic drugs for the management of moderate to severe pain. Control of the rate and duration of drug delivery is particularly important for transdermal delivery of analgesic drugs to avoid the potential risk of overdose and the discomfort of an insufficient dosage.

One class of analgesics that has found application in a transdermal delivery route is the synthetic opiates, a group of 4-aniline piperidines. The synthetic opiates, e.g., fentanyl and certain of its derivatives such as sufentanil, are particularly well-suited for transdermal administration. These synthetic opiates are characterized by their rapid onset of analgesia, high potency, and short duration of action. They are estimated to be 80 and 800 times, respectively, more potent than morphine. These drugs are weak bases, i.e., amines, whose major fraction is cationic in acidic media.

In an in vivo study to determine plasma concentration, Thysman and Preat (Anesth. Analg. 77 (1993) pp. 61-66) compared simple diffusion of fentanyl and sufentanil to electrotransport delivery in citrate buffer at pH 5. Simple diffusion did not produce any detectable plasma concentration. The plasma levels attainable depended on the maximum flux of the drug that can cross the skin and the drug's pharmacokinetic properties, such as clearance and volume of distribution. Electrotransport delivery was reported to have significantly reduced lag time (i.e., time required to achieve peak plasma levels) as compared to passive transdermal patches (1.5 h versus 14 h). The researchers' conclusions were that electrotransport of these analgesic drugs can provide more rapid control of pain than classical patches, and a pulsed release of drug (by controlling electrical current) was comparable to the constant delivery of classical patches. See, also, e.g., Thysman et al. Int. J. Pharm., 101 (1994) pp. 105-113; V. Preat et al. Int. J. Pharm., 96 (1993) pp. 189-196 (sufentanil); Gourlav et al. Pain, 37 (1989) pp. 193-202 (fentanyl); Sebel et al. Eur. J. Clin. Pharmacol., 32 (1987) pp. 529-531 (fentanyl and sufentanil).

Passive, i.e., by diffusion, and electrically-assisted transdermal delivery of narcotic analgesic drugs, such as fentanyl and sufentanil, to induce analgesia, have also both been described in the patent literature. See, e.g., Gale et al., U.S. Pat. No. 4,588,580, Aungst et al., U.S. Pat. No. 4,626,539, Levy et al., U.S. Pat. No. 4,822,802, Cleary et al., U.S. Pat. No. 4,906,463, Theeuwes et al., U.S. Pat. No. 5,232,438, Gevirtz et al., U.S. Pat. No. 5,635,204, Southam et al., U.S. Pat. No. 6,171,294, Southam et al., U.S. Pat. No. 6,216,033, Southam et al., U.S. Pat. No. 6,425,892, Phipps et al., U.S. Pat. No. 6,881,208, Southam et al., U.S. Pat. Pub. No. US 2003/0083609, Venkatraman et al., U.S. Pat. Pub. No. US 2003/0026829, Venkatraman et al., U.S. Pat. Pub. No. US 2004/0213832, Phipps et al., U.S. Pat. Pub. No. US 2005/0131337.

Another fentanyl derivative, lofentanil, is reported to be 20-30 times more potent than fentanyl (see, e.g., Mather, Clin. Pharmacokinet., 8 (1983) pp. 422-446; Dosen-Micovic, J. Serb. Chem. Soc., 69 (2004) pp. 843-854). Carfentanil is in the same potency range as lofentanil. As such, lofentanil and carfentanil have an advantage over fentanyl in the treatment of pain. To obtain the same analgesic effect, less drug is necessary, resulting in fewer side effects. However, due to the fact that lofentanil and carfentanil are both 20-30 times more potent that fentanyl, the chances of an accidental overdose are greater, which can result in respiratory depression and other adverse side effects. In addition, the substitution of lofentanil or carfentanil or any other opioid in a drug delivery device is not necessarily a straightforward process, and consideration must be given to issues such as stability of the opioid and shelf life in a packaged system, particularly an aqueous system.

Although passive transdermal delivery of lofentanil and carfentanil have been described, e.g., Levy et al., U.S. Pat. No. 4,822,802, Gevirtz et al., U.S. Pat. No. 5,635,204, Venkatraman et al., U.S. Pat. Pub. No. US 2003/0026829, Venkatraman et al., U.S. Pat. Pub. No. US 2004/0213832, there is a need for lofentanil and carfentanil formulations in a suitable electrotransport device to take advantage of the convenience of electrotransport delivery in a small, self-contained, patient-controlled device. In addition, there is a need to provide systems and devices capable of accurately delivering the required dosage of lofentanil and carfentanil without the danger of overdosage. Furthermore, it would be desirable to provide an electrotransport device and system that is stable and has an acceptable shelf life.

### SUMMARY OF THE INVENTION

Embodiments of the present invention provide systems, methods and devices for transdermal electrotransport delivery of lofentanil or carfentanil. As such, according to an embodiment of the present invention, a device designed for electrotransport delivery of lofentanil or carfentanil is provided, concomitantly providing a greater measure of patient safety and comfort in pain management than previously achieved by other opioids. In one or more embodiments, lofentanil or carfentanil is delivered through a body surface (e.g., intact skin) by an electrotransport device, the device having an anodic donor reservoir containing an at least partially aqueous solution of a lofentanil or carfentanil salt. Because less drug is necessary to achieve a suitable analgesic effect, a smaller electrotransport device can be used to deliver lofentanil than previously used to deliver other opiates.

Embodiments of the present invention further relate to devices, systems and methods for administering lofentanil or carfentanil by transdermal electrotransport to treat acute, chronic and/or breakthrough pain. A transdermal electrotransport dose of about 0.5 to 5 μg of lofentanil or carfentanil, delivered over a delivery interval of up to about 20 minutes, is therapeutically effective in treating acute post-operative pain in human patients having body weights above about 35 kg. Preferably, the amount of lofentanil or carfentanil delivered is about 1 μg to about 3 μg over a delivery interval of about 5 to 15 minutes, and most preferably the amount of lofentanil or carfentanil delivered is about 2 μg over a delivery interval of about 10 minutes.

A transdermal electrotransport dose of about 0.3 μg/hr to about 10 μg/hr of lofentanil or carfentanil, delivered over a delivery interval of up to about 7 days, is therapeutically effective in treating chronic, baseline pain in human patients having body weights above about 35 kg. Preferably, the amount of lofentanil or carfentanil delivered is about 1 μg/hr to about 5 μg/hr over a delivery interval of about 1 to 7 days, and most preferably the amount of lofentanil or carfentanil delivered is about 2 to 4 μg/hr over a delivery interval of about 3 days.

A transdermal electrotransport dose of about 3 μg to about 40 μg of lofentanil or carfentanil, delivered over a delivery interval of up to about 20 minutes, is therapeutically effective in treating breakthrough pain in human patients having body weights above about 35 kg. Preferably, the amount of lofentanil or carfentanil delivered is about 10 μg to about 20 μg over a delivery interval of about 5 to 15 minutes, and most preferably the amount of lofentanil or carfentanil delivered is about 15 μg over a delivery interval of about 10 minutes.

The device for transdermally delivering lofentanil or carfentanil by electrotransport may further include means for delivering at least 1 additional, and more preferably about 10 to 100 additional like dose(s) of lofentanil or carfentanil over subsequent like delivery period(s) over about 24 hours. For example, up to about 100 additional like doses of lofentanil or carfentanil may be used to treat acute post-operative pain. Likewise, up to about 10 additional like doses per day may be used to treat breakthrough pain. The ability to deliver multiple identical doses from a transdermal electrotransport lofentanil or carfentanil delivery device also provides the capability of pain management to a wider patient population, in which different patients require different amounts of lofentanil or carfentanil to control their pain. By providing the capability of administering multiple small transdermal electrotransport lofentanil or carfentanil doses, the patients can titrate themselves to administer only that amount of lofentanil or carfentanil which is needed to control their pain, and no more.

Other advantages and a fuller appreciation of specific adaptations, compositional variations, and physical attributes of the present invention can be learned from an examination of the following drawings, detailed description, examples, and appended claims.

### BRIEF DESCRIPTION OF THE DRAWINGS

The present invention is hereinafter described in conjunction with the appended drawings, in which:

FIG. 1 is a perspective exploded view of an electrotransport drug delivery device in accordance with the present invention.

### DETAILED DESCRIPTION OF THE INVENTION

Embodiments of the present invention provide a lofentanil or carfentantil salt electrotransport delivery device, and a method of using same, to achieve a systemic analgesic effect. One embodiment of the present invention provides an electrotransport delivery device for delivering lofentanil or carfentanil through a body surface, e.g., skin, to achieve the analgesic effect. The lofentanil or carfentanil salt is provided in a donor reservoir of an electrotransport delivery device, preferably as an aqueous salt solution.

In one embodiment, the dose of lofentanil or carfentanil delivered by transdermal electrotransport for treating acute post-operative pain is about 0.5 μg to about 5 μg, delivered over a period of up to about 20 minutes. Preferred is a dosage of about 1 μg to about 3 μg delivered over a period of about 5 to about 15 minutes, and most preferred is a dosage of about 2 μg for a delivery period of about 10 minutes.

In another embodiment, the dose of lofentanil or carfentanil delivered by transdermal electrotransport for treating chronic, baseline pain is about 0.3 μg/hr to about 10 μg/hr, delivered over a period of up to about 7 days. Preferred is a dosage of about 1 μg/hr to about 5 μg/hr, and most preferred is a dosage range of about 2 μg/hr to 4 ug/hr for the delivery period.

According to a further embodiment, the dose of lofentanil or carfentanil delivered by transdermal electrotransport for treating breakthrough pain is about 3 μg to about 40 μg, delivered over a period of up to about 20 minutes. Preferred is a dosage of about 10 μg to about 20 μg delivered over a period of time of about 5 to 15 minutes, and most preferred is a dosage of about 15 μg for a delivery period of about 10 minutes.

According to one or more embodiments of the invention, the device further preferably includes means for delivering about 10 to 100 additional like doses over a period of 24 hours in order to achieve and maintain the analgesic effect for treating acute post-operative pain. For example, up to about 100 additional like doses of lofentanil or carfentanil may be used to treat acute post-operative pain, while up to about 10 additional like doses may be used to treat breakthrough pain.

The lofentanil or carfentanil salt-containing anodic reservoir formulation for transdermally delivering the above mentioned doses of lofentanil or carfentanil by electrotransport is preferably comprised of an aqueous solution of a water soluble lofentanil or carfentanil salt such as HCl, oxalate or citrate salts. Methods for the manufacture of lofentanil or carfentanil and its pharmaceutically acceptable acid addition salts are well known in the art (see, e.g., Janssen et al., U.S. Pat. No. 3,998,834). Most preferably, the aqueous solution is contained within a hydrophilic polymer matrix such as a hydrogel matrix. The lofentanil or carfentanil salt is present in an amount sufficient to deliver the above mentioned doses transdermally by electrotransport over the defined delivery periods to achieve the desired analgesic effect, as described further below.

The anodic lofentanil or carfentanil salt-containing hydrogel can suitably be made of any number of materials but preferably is comprised of a hydrophilic polymeric material, preferably one that is polar in nature so as to enhance the drug stability. Suitable polar polymers for the hydrogel matrix comprise a variety of synthetic and naturally occurring polymeric materials. A preferred hydrogel formulation contains a suitable hydrophilic polymer, a buffer, a humectant, a thickener, water and a water soluble lofentanil or carfentanil salt (e.g., HCl salt). A preferred hydrophilic polymer matrix is polyvinyl alcohol such as a washed and fully hydrolyzed polyvinyl alcohol (PVOH), e.g., Mowiol 66-100, commercially available from Hoechst Aktiengesellschaft. A suitable buffer is an ion exchange resin which is a copolymer of methacrylic acid and divinylbenzene in both an acid and salt form. One example of such a buffer is a mixture of Polacrilin (the copolymer of methacrylic acid and divinyl benzene available from Rohm & Haas, Philadelphia, Pa.) and the potassium salt thereof. A mixture of the acid and potassium salt forms of Polacrlin functions as a polymeric buffer to adjust the pH of the hydrogel to between about pH 4 and about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g., Methocel KOOMP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

In one preferred embodiment, the anodic lofentanil or carfentanil salt-containing hydrogel formulation comprises about 15 to 20 wt % polyvinyl alcohol, and about 1 to 2.5 wt % lofentanil or carfentanil salt, preferably the hydrochloride salt. The remainder is water and ingredients such as humectants, thickeners, etc. The polyvinyl alcohol (PVOH)-based hydrogel formulation is prepared by mixing all materials, including the lofentanil or carfentanil salt, in a single vessel at elevated temperatures of about 90° C. to 95° C. for at least about 0.5 hr. The hot mix is then poured into foam molds and stored at freezing temperature of about −35° C. overnight to cross-link the PVOH. Upon warming to ambient temperature, a tough elastomeric gel is obtained suitable for lofentanil or carfentanil electrotransport.

As is known in the art, there are several concerns associated with prefilled devices, such as storage. Many drugs have poor stability when in solution. Accordingly, the shelf life of prefilled iontophoretic drug delivery devices may be unacceptably short. Corrosion of the electrodes and other electrical components is also a potential problem with prefilled devices. For example, the return electrode assembly will usually contain an electrolyte salt such as sodium chloride which over time can cause corrosion of metallic and other electrically conductive materials in the electrode assembly. Leakage is another serious problem with prefilled iontophoretic drug delivery devices. Leakage of drug or electrolyte from the electrode receptacle can result in an inoperative or defective state.

Thus, it may be desirable to provide an electrotransport system having dry electrodes that are hydratable. Examples of such dry state electrode devices are disclosed in commonly assigned U.S. Pat. Nos. 6,374,136, 5,582,587, 5,533,972, 5,385,543, 5,320,598, 5,310,404, 5,288,289 and 5,158,537, the entire contents of each of these patents being incorporated herein by reference.

The hydrogel formulations are used in an electrotransport device such as described hereinafter. A suitable electrotransport device includes an anodic donor electrode, preferably comprised of silver, and a cathodic counter electrode, preferably comprised of silver chloride. The donor electrode is in electrical contact with the donor reservoir containing the aqueous solution of a lofentanil or carfentanil salt. As described above, the donor reservoir is preferably a hydrogel formulation. The counter reservoir also preferably comprises a hydrogel formulation containing a (e.g., aqueous) solution of a biocompatible electrolyte, such as citrate buffered saline. The anodic and cathodic hydrogel reservoirs preferably each have a skin contact area of about 0.1 to about 20 cm² and more preferably about 0.2 to 10 cm². The anodic and cathodic hydrogel reservoirs preferably have a thickness of about 0.01 to 0.4 cm, and more preferably about 0.05 cm.

It will be appreciated that the skin contact area and gel thickness will depend on the particular application of the device, namely whether the device will be used to treat acute pain, chronic pain or in breakthrough pain applications. Each of these applications will require a different dosage amount and dosage rate, and as is known by those skilled in the art of electrotransport delivery, factors including efficiency (measured in μg/μA/h and determined experimentally), current, contact area, current density, gel thickness, drug utilization fraction, drug concentration and passive flux will determine the desired delivery rate of the drug. Therefore, by employing calculations known in the art and target ranges for various device attributes, the device parameters can be determined. A desired target range for the efficiency is between about 0.1 to 1.4 μg/μA/h and can be determined experimentally. The duration of delivery will depend on the particular application. For acute and breakthrough pain applications, a desired duration of delivery is between about 1 minute and 20 minutes, and for chronic pain, a desired duration of delivery is between about 1 day and 7 days or more. A desired contact area is between about 0.3 and 10 cm². A desired current density is between about 10 and 200 μA/cm², and a desired gel thickness is between about 0.02 and 0.5 cm. A desired range for drug utilization can be experimentally determined and typically should be between about 0.1 and 0.7. The drug concentration typically is desired to between about 10 to 25 mg/ml.

The applied electrotransport current is about 0.1 μA to about 2400 μA, depending on the analgesic effect desired. Most preferably, the applied electrotransport current is substantially constant DC current during the dosing interval. The appropriate current can be determined using calculations known in the art.

The passive flux of the drug can be experimentally determined. Since the passive flux is expected to be between about 0.5 to 3 μg/h/cm², it may be desirable to utilize one or more flux control membranes to minimize the passive flux rate of the drug. For example, flux control membranes such as those disclosed in Theeuwes et al., U.S. Pat. Nos. 5,080,646; 5,147,296; 5,169,382; 5,169,383; 5,322,502; and 6,163,720, can be positioned between the donor reservoir and the body surface of the patient and between the counter reservoir and the body surface of the patient, respectively, in order to limit or control the amount of passive, i.e. non-electrically assisted, flux of agent to the body surface. Each of these patents disclosing flux control agents are incorporated herein by reference in their entirety. The membranes can be made from various materials such as hydrophobic polymers and hydrophilic polymers. Exemplary hydrophobic polymers include polycarbonates, polyisobutylenes, polyethylenes, polypropylenes, polyisoprenes, polyalkenes, rubbers, polyvinylacetates, ethylene vinyl acetate copolymers, polyamides, nylons, polyurethanes, polyvinylchlorides; acrylic or methacrylic acid esters of an alcohol such as n-butanol, 1-methyl pentanol, 2-methyl pentanol, 3-methyl pentanol, 2-ethyl butanol, iso-octanol, n-decanol, and combinations thereof; such acrylic or methacrylic acid esters of an alcohol copolymerized with one or more ethylenically unsaturated monomers such as acrylic acid, methacrylic acid, acrylamides, methacrylamides, n-alkoxymethyl acrylamides, n-alkoxymethyl methacrylamides, n-tert-butylacrylamides, itaconic acid, n-branched alkyl maleamic acids having 10-24 carbons in the alkyl group, glycol diacrylates, and mixtures and combinations thereof. It is also preferred that the hydrophobic or hydrophilic polymer used to make the low and/or high porosity membrane be heat fusible. Examples of hydrophilic polymers include copolyesters, polyvinylpyrrolidones, polyvinyl alcohols, polyethylene oxides, blends of polyethylene oxides or polyethylene glycols with polyacrylic acid, polyacrylamides, crosslinked dextran, starch grafted poly(sodium acrylate-co-acrylamides, cellulose derivatives (such as hydroxyethyl celluloses, hydroxypropylmethyl celluloses, low-substituted hydroxypropyl celluloses, and crosslinked sodium carboxymethyl celluloses such as Ac-Di-Sol from FMC Corp. of Philadelphia, Pa), hydrogels (such as polyhydroxylethyl methacrylates available from National Patent Development Corp.), natural gums, chitosans, pectins, starches, guar gums, locust bean gums, blends and combinations thereof, and equivalent materials thereof.

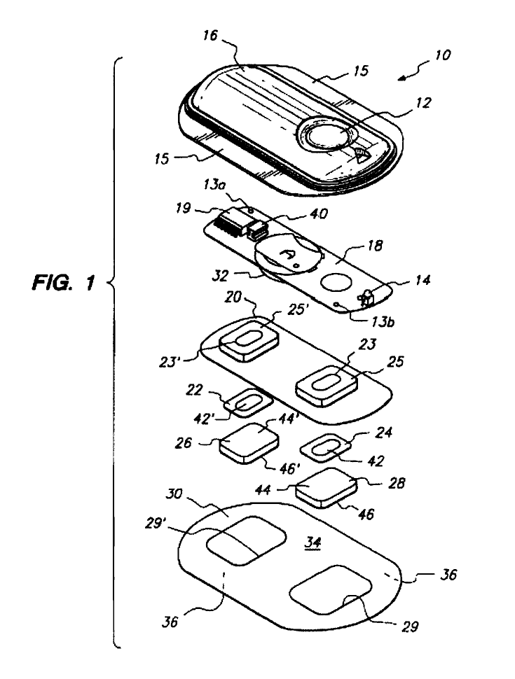

Reference is now made to FIG. 1 which depicts an exemplary electrotransport device which can be used in accordance with the present invention. FIG. 1 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anode electrode 22, cathode electrode 24, anode reservoir 26, cathode reservoir 28 and skin-compatible adhesive 30. Upper housing 16 has lateral wings 15 which assist in holding device 10 on a patient's skin. Upper housing 16 is preferably composed of an injection moldable elastomer (e.g., ethylene vinyl acetate). Printed circuit board assembly 18 comprises an integrated circuit 19 coupled to discrete electrical components 40 and battery 32. Circuit board assembly 18 is attached to housing 16 by posts (not shown in FIG. 1) passing through openings 13a and 13b, the ends of the posts being heated/melted in order to heat stake the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15. Shown (partially) on the underside of circuit board assembly 18 is a battery 32, which is preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23, 23′ in the depressions 25, 25′ formed in lower housing 20, by means of electrically conductive adhesive strips 42, 42′. Electrodes 22 and 24, in turn, are in direct mechanical and electrical contact with the top sides 44′, 44 of reservoirs 26 and 28. The bottom sides 46′, 46 of reservoirs 26, 28 contact the patient's skin through the openings 29′, 29 in adhesive 30. Upon depression of push button switch 12, the electronic circuitry on circuit board assembly 18 delivers a predetermined DC current to the electrodes/reservoirs 22, 26 and 24, 28 for a delivery interval of predetermined length, e.g., about 10 minutes. Preferably, the device transmits to the user a visual and/or audible confirmation of the onset of the drug delivery, or bolus, interval by means of LED 14 becoming lit and/or an audible sound signal from, e.g., a “beeper”. Lofentanil or carfentanil is then delivered through the patient's skin, e.g., on the arm, for the predetermined (e.g., 10 minute) delivery interval. In practice, a user receives feedback as to the onset of the drug delivery interval by visual (LED 14 becomes lit) and/or audible signals (a beep from the “beeper”).

Anodic electrode 22 is preferably comprised of silver and cathodic electrode 24 is preferably comprised of silver chloride. Both reservoirs 26 and 28 are preferably comprised of polymer hydrogel materials as described herein. Electrodes 22, 24 and reservoirs 26, 28 are retained by lower housing 20. For lofentanil or carfentanil salts, the anodic reservoir 26 is the “donor” reservoir which contains the drug and the cathodic reservoir 28 contains a biocompatible electrolyte.

The push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is preferably composed of a plastic or elastomeric sheet material (e.g., polyethylene) which can be easily molded to form depressions 25, 25′ and cut to form openings 23, 23′. The assembled device 10 is preferably water resistant (i.e., splash proof) and is most preferably waterproof. The system has a low profile that easily conforms to the body thereby allowing freedom of movement at, and around, the wearing site. The anode/drug reservoir 26 and the cathode/salt reservoir 28 are located on the skin-contacting side of device 10 and are sufficiently separated to prevent accidental electrical shorting during normal handling and use.

The device 10 adheres to the patient's body surface (e.g., skin) by means of a peripheral adhesive 30 which has upper side 34 and body-contacting side 36. The adhesive side 36 has adhesive properties which assures that the device 10 remains in place on the body during normal user activity, and yet permits reasonable removal after the predetermined (e.g., 24-hour) wear period. Upper adhesive side 34 adheres to lower housing 20 and retains the electrodes and drug reservoirs within housing depressions 25, 25′ as well as retains lower housing 20 attached to upper housing 16.

The push button switch 12 is located on the top side of device 10 and is easily actuated through clothing. A double press of the push button switch 12 within a short period of time, e.g., three seconds, is preferably used to activate the device 10 for delivery of drug, thereby minimizing the likelihood of inadvertent actuation of the device 10.

Upon switch activation, an audible alarm signals the start of drug delivery, at which time the circuit supplies a predetermined level of DC current to the electrodes/reservoirs for a predetermined (e.g., 10 minute) delivery interval. The LED 14 remains “on” throughout the delivery interval indicating that the device 10 is in an active drug delivery mode. The battery preferably has sufficient capacity to continuously power the device 10 at the predetermined level of DC current for the entire (e.g., 24 hour) wearing period.

Since lofentanil or carfentanil are bases, the salts of lofentanil or carfentanil are typically acid addition salts, e.g., citrate salts, hydrochloride salts, oxalate salts, etc. When these salts are placed in solution (e.g., aqueous solution), the salts dissolve and form protonated lofentanil or carfentanil cations and counter (e.g., citrate, chloride, oxalate) anions. As such, the lofentanil or carfentanil cations are delivered from the anodic electrode of an electrotransport delivery device. Silver anodic electrodes have been proposed for transdermal electrotransport delivery as a way to maintain pH stability in the anodic reservoir. See, e.g., Untereker et al. U.S. Pat. No. 5,135,477 and Petelenz et al. U.S. Pat. No. 4,752,285. These patents also recognize one of the shortcomings of using a silver anodic electrode in an electrotransport delivery device, namely that the application of current through the silver anode causes the silver to become oxidized (Ag→Ag⁺+e⁻), thereby forming silver cations which compete with the cationic drug for delivery into the skin by electrotransport. Silver ion migration into the skin results in a transient epidermal discoloration (TED) of the skin. In accordance with the teachings in these patents, the cationic lofentanil or carfentanil is preferably formulated as a halide salt (e.g., hydrochloride salt) so that any electrochemically-generated silver ions will react with the drug counter ions (i.e., halide ions) to form a substantially insoluble silver halide (Ag⁺+X⁻→AgX). In addition to these patents, Phipps et al. WO 95/27530 teaches the use of supplementary chloride ion sources in the form of high molecular weight chloride resins in the donor reservoir of a transdermal electrotransport delivery device. These resins are highly effective at providing sufficient chloride for preventing silver ion migration, and the attendant skin discoloration when delivering lofentanil or carfentanil by electrotransport using a silver anodic electrode.

In summary, the embodiments of present invention provide apparatus, devices, systems and methods for the transdermal electrotransport of water soluble salts of lofentanil or carfentanil, which are preferably delivered from an electrotransport device having a silver anodic donor electrode and a hydrogel based donor reservoir. The electrotransport device is preferably a patient-controlled device. The hydrogel formulation contains a drug concentration which is sufficient to maintain transdermal electrotransport drug flux for a predetermined current level, to inhibit silver ion migration to the skin of a wearer of the electrotransport device and thus, prevent transient epidermal discoloration, and to provide an acceptable level of analgesia.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method of obtaining analgesia in a human patient who is suffering from pain consisting of transdermally delivering solely by electrotransport a dose of about 0.5 μg to about 5 μg of lofentanil or carfentanil from an electrotransport device over a predetermined delivery period of up to about 20 minutes, terminating said delivery at the end of said delivery period and thereafter repeating such transdermal administering up to about 100 additional of said doses over a period of 24 hours.

2. The method of claim 1, wherein the delivery period is about 10 minutes.

3. The method of claim 1, where the lofentanil or carfentanil comprises a lofentanil or carfentanil salt.

4. The method of claim 1, wherein the electrotransport device comprises a donor reservoir hydrogel formulation.

5. The method of claim 4, wherein the donor reservoir hydrogel formulation comprises about 1 to about 2.5 weight % lofentanil or carfentanil salt.

6. A method of obtaining analgesia in a human patient who is suffering from pain consisting of transdermally delivering solely by electrotransport a dose of about 0.3 μg/hr to about 10 μg/hr of lofentanil or carfentanil from an electrotransport device over a delivery period of up to about 7 days.

7. The method of claim 6, where the lofentanil or carfentanil comprises a lofentanil or carfentanil salt.

8. The method of claim 1, wherein the electrotransport device comprises a donor reservoir hydrogel formulation.

9. The method of claim 4, wherein the donor reservoir hydrogel formulation comprises about 1 to about 2.5 weight % lofentanil or carfentanil salt.

10. A method of obtaining analgesia in a human patient who is suffering from pain consisting of transdermally delivering solely by electrotransport a dose of about 3 μg to about 40 μg of lofentanil or carfentanil from an electrotransport device over a predetermined delivery period of up to about 20 minutes, terminating said delivery at the end of said delivery period and thereafter repeating such transdermal administering up to about 10 additional of said doses over a period of 24 hours.

11. The method of claim 10, wherein the delivery period is about 10 minutes.

12. The method of claim 10, where the lofentanil or carfentanil comprises a lofentanil or carfentanil salt.

13. The method of claim 10, wherein the electrotransport device comprises a donor reservoir hydrogel formulation.

14. The method of claim 13, wherein the donor reservoir hydrogel formulation comprises about 1 to about 2.5 weight % lofentanil or carfentanil salt.

15. A method of obtaining analgesia in a human patient who is suffering from pain consisting of transdermally delivering solely by electrotransport a dose of lofentanil or carfentanil from an electrotransport device sufficient to alleviate said pain.

16. A device for transdermally delivering lofentanil or carfentanil by electrotransport, comprising a donor reservoir containing lofentanil or carfentanil in a form to be delivered solely by electrotransport, a counter reservoir, a source of electrical power electrically connected to said reservoirs and a control circuit for controlling the magnitude and timing of applied electrotransport current.

17. The device of claim 16, wherein the reservoirs, the power source and the control circuit are configured to deliver by electrotransport about 0.5 μg to about 20 μg of lofentanil or carfentanil over a delivery period of up to about 20 minutes.

18. The device of claim 16, where the lofentanil or carfentanil comprises a lofentanil or carfentanil salt.

19. The device of claim 16, wherein the donor reservoir comprises a hydrogel formulation.

20. The device of claim 19, further comprising a membrane located on the body surface distal side of the device.
